(19) United States
(12) Patent Application Publication
(10) Pub. No.: US 2026/0169004 A1
(43) Pub. Date: Jun. 18, 2026

(54) ANTIBODY DISCOVERY THROUGH LONGITUDINAL LINEAGE TRACING

(71) Applicants: ZHEJIANG NANOMAB TECHNOLOGY CENTER CO. LTD., SHAOXING, ZHEJIANG (CN); SHANGHAI CHENGHUANG NANOMAB TECHNOLOGY CO. LTD., Jiading District, Shanghai (CN)

(72) Inventors: Xinhao WANG, Shanghai (CN); Jiaguo LI, Shanghai (CN); Weimin ZHU, Shanghai (CN)

(21) Appl. No.: 19/127,086

(22) PCT Filed: Nov. 2, 2023
(86) PCT No.: PCT/CN2023/129234

§ 371 Date: May 2, 2025

Related U.S. Application Data
(60) Provisional application No. 63/382,105, filed on Nov. 2, 2022.

(51) Int. Cl. G01N 33/68; C12N 15/10

(52) U.S. Cl. CPC G01N 33/6845; C12N 15/1037

(57) ABSTRACT

A method of generating an antibody specific for a target antigen, the method comprising (a) subject an animal to one or more rounds of immunization with the target antigen; (b) collecting a first sample of B cells from the animal; (c) identifying one or more first antibodies from a B cell in the first sample that specifically bind to the antigen and determining a genetic element characterizing the B cell lineage of the B cell in the first sample; (d) subjecting the animal to one or more additional rounds of immunization with the target antigen; (e) collecting a second sample of B cells from the animal; (f) identifying one or more second antibodies from a B cell in the second sample that is from the same B cell lineage as the B cell in the first sample; and (g) testing the second antibodies for specific binding to the target antigen. optionally, In certain embodiments, steps (d) through (g) are repeated.

### CROSS-REFERENCE TO RELATED APPLICATIONS

This application claims the benefit of U.S. provisional application No. 63/382,105, filed Nov. 2, 2022, which is incorporated by reference in its entirety.

### BACKGROUND OF THE INVENTION

### (1) Field of the Invention

The present application generally relates to antibody discovery. More specifically, the application is directed to the discovery of antibodies in the same lineage.

### (2) Description of the Related Art

Antibodies have been used in many applications where high affinity and specificity are critical properties for their practical applications. In the immune system, antibodies are produced by B cells. After encountering antigens, naïve B cells expand and proliferate and go through multiple rounds of somatic hypermutation (SHM) and/or gene conversion and positive/negative selections in the germinal center to generate diverse set of B cells which produce antibodies with higher affinity and specificity against a target antigen than antibodies earlier in the B cell lineage (FIG. 1; Küppers R., 2005). This in vivo affinity maturation process generally increases antibody activity as it accumulates more and more mutations in the antibody sequence. See, e.g., Jiang et al., 2013; Kepler et al., 2014; Zhang et al., 2016; Pan et al., 2023; U.S. Pat. Nos. 7,117,096 B2; 7,432,063 B2; 10,101,333 B2; PCT Patent Application Publication No. WO 2020/176815. However, the underlining cellular and molecular mechanisms of the process remain poorly characterized.

In addition, in vivo affinity maturation and other non-bias screening methods can reduce the developability issue which is usually resulted from conventional phage-display selection process (Daniela Bumbaca et al., 2011).

High throughput sequencing technologies such as next generation sequencing (NGS) technology have been extensively used as an effective sampling method to analyze immune repertoires. These technologies can sequence millions of B cells to capture a snapshot of B cell repertoire at specific time points, thus providing an effective and cost-efficient sampling method for studying the dynamics of immune repertoires and tracing longitudinal lineage changes (DeKosky et al., 2013; Phad et al., 2022).

The present invention exploits the affinity maturation process captured by high throughput sequencing technologies in immunized animals to obtain high affinity antibodies to an antigen from B cell clonal lineages that produced antigen-binding antibodies earlier in the lineage.

### BRIEF SUMMARY OF THE INVENTION

Provided is a method of generating an antibody by immunizing an animal with an antigen over time, repeatedly isolating B cells from the animal, and identifying antibodies in later-isolated B cells that are in the same lineage as earlier-isolated B cells that produce antibodies that bind to the antigen.

Thus, in some embodiments, a method of generating an antibody specific for a target antigen is provided. The method comprises
(a) subject an animal to one or more rounds of immunization with the target antigen;
(b) collecting a first sample of B cells from the animal;
(c) identifying one or more first antibodies from a B cell in the first sample that specifically bind to the antigen and determining a genetic element characterizing the B cell lineage of the B cell in the first sample;
(d) subjecting the animal to one or more additional rounds of immunization with the target antigen;
(e) collecting a second sample of B cells from the animal;
(f) identifying one or more second antibodies from a B cell in the second sample that is from the same B cell lineage as the B cell in the first sample; and
(g) testing the second antibodies for specific binding to the target antigen.

The additional rounds of immunization, collecting B cells, and identifying and testing antibodies in steps (d) through (g) may be repeated one or more times.

### BRIEF DESCRIPTION OF THE SEVERAL VIEWS OF THE DRAWINGS

FIG. 1 is an illustration of a germinal center and the B cell development therein from Küppers R., 2005.

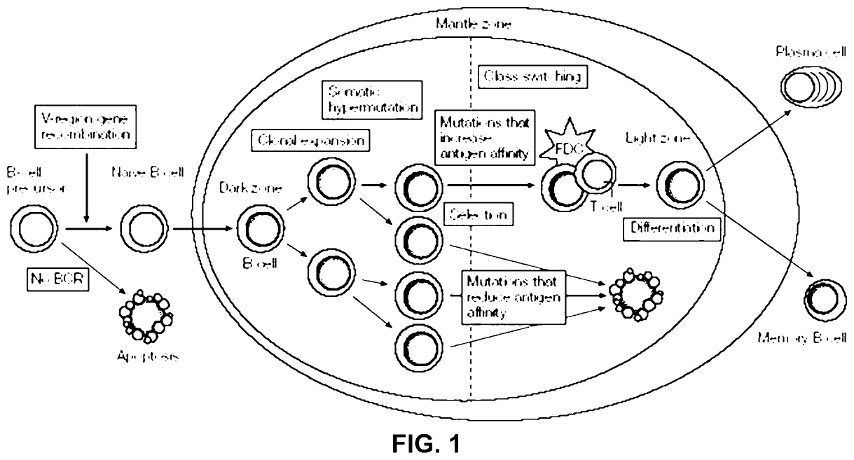

FIG. 2 is an illustration of an exemplary timeline and steps for performing the method described herein.

FIG. 3 is an exemplary flow chart of processing and analysis of NGS data generated from libraries from PBMC samples from an animal immunized multiple times with a target antigen.

FIG. 4 is an exemplary flow chart for processing and selecting antibodies for testing.

FIG. 5 shows an exemplary map of antibody domains, with PCR primers (arrows) to cover partial CDR3 sequences of antibodies to enrich specific lineage groups.

FIG. 6 is a graph of lineage groups and clone mapping data from an NGS dataset (ALP07VHH_PB_PAN) after the 7^{th} immunization.

FIGS. 7A and 7B are graphs showing an increase of mismatch scores with further immunization. FIG. 7A is from an NGS dataset (ALP07VHH_PB_PAN) generated after the 7^{th} immunization. The average mismatch score for the dataset is 8.49. FIG. 7B is from an NGS dataset (NBL504-A27L1P1L-R2) generated after the 24^{th} immunization. The average mismatch score for the dataset is 15.524. The difference of mismatch scores between these two datasets is significant (P<0.001, T-test).

FIG. 8 is a graph showing lineage group mapping between NGS datasets. ALP07VHH_PB_PAN is mapped to NBL504-A27L1P1L-R2. Twenty eight lineage groups are shared between these two NGS datasets which have 2518 and 2341 lineage groups respectively.

FIG. 9 shows the immunization and bleeding schedules for an alpaca A050 and the corresponding 7 PBMC samples used in the analysis.

FIG. 10 is a graph showing titers of the 7 PBMC samples.

FIG. 11 is a graph showing average mismatch scores of NGS libraries constructed using the 7 PBMC samples.

FIG. 12 is a graph showing the average mismatch scores of shared lineages among repertoires of the 7 PBMC samples.

FIG. 13 is a graph showing the dynamics of mismatch scores for three antigen specific lineages.

FIG. 14 is a graph showing the correlation between mismatch scores and ELISA value for binders from alpaca A050.

FIG. 15 is a graph showing the correlation between mismatch scores and ELISA value for binders from a llama.

### DETAILED DESCRIPTION OF THE INVENTION

### Definitions

The term “plurality” refers to more than 1, for example more than 2, more than about 5, more than about 10, more than about 20, more than about 50, more than about 100, more than about 200, more than about 500, more than about 1000, more than about 2000, more than about 5000, more than about 10,000, more than about 20,000, more than about 50,000, more than about 100,000, usually no more than about 200,000. A “population” contains a plurality of items.

The term “epitope” as used herein can include any protein determinant capable of specific binding to an immunoglobulin or T-cell receptor. Epitopic determinants usually consist of chemically active surface groupings of molecules such as amino acids or sugar side chains and usually have specific three-dimensional structural characteristics, as well as specific charge characteristics. An antibody is said to specifically bind an antigen when the equilibrium dissociation constant is ≤1 μM, preferably ≤100 nM and most preferably ≤10 nM.

The term “K_{D}” refers to the equilibrium dissociation constant of a particular antibody-antigen interaction.

The term “immune response” as used herein can refer to the action of, for example, lymphocytes, antigen presenting cells, phagocytic cells, granulocytes, and soluble macromolecules produced by the above cells or the liver (including antibodies, cytokines, and complement) that results in selective damage to, destruction of, or elimination from an organism of invading pathogens, cells or tissues infected with pathogens, cancerous cells, or, in cases of autoimmunity or pathological inflammation, normal organismal cells or tissues.

As used herein, the term “antibody” refers to (a) an intact immunoglobulin, (b) a monoclonal or polyclonal antigen-binding fragment with or without the Fc (crystallizable fragment) region or FcRn binding fragment of the Fc region (“Fc fragment” or “Fc region”), (c) a nanobody (including naturally occurring camelid nanobodies and heavy chain only (“VHH”) antibodies) or antigen-binding fragment thereof, or (d) an IgNAR antibody found in sharks and other elasmobranchs. The antigen-binding fragments may be produced by recombinant DNA techniques or by enzymatic or chemical cleavage of intact antibodies. Antigen-binding fragments include, inter alia, Fab, Fab′, F(ab′)2, Fv, dAb, and complementarity determining region (CDR) fragments, single-chain antibodies (scFv), single region antibodies, chimeric antibodies, CDR grafted antibodies, humanized antibodies, biparatopic antibodies, diabodies and polypeptides that contain at least a portion of an immunoglobulin that is sufficient to confer specific antigen binding to the polypeptide. The Fc region includes portions of two heavy chains contributing to two or three classes of the antibody. The Fc region may be produced by recombinant DNA techniques or by enzymatic (e.g. papain cleavage) or via chemical cleavage of intact antibodies.

The term “antibody fragment,” as used herein, refers to a protein fragment that comprises only a portion of an intact antibody, generally including an antigen binding site of the intact antibody and thus retaining the ability to bind antigen. Examples of antibody fragments encompassed by the present definition include: (i) the Fab fragment, having VL, CL, VH and CH1 regions; (ii) the Fab′ fragment, which is a Fab fragment having one or more cysteine residues at the C-terminus of the CH1 region; (iii) the Fd fragment having VH and CH1 regions; (iv) the Fd′ fragment having VH and CH1 regions and one or more cysteine residues at the C-terminus of the CH1 region; (v) the Fv fragment having the VL and VH regions of a single arm of an antibody; (vi) the dAb fragment (Ward et al., 1989) which consists of a VH region; (vii) isolated CDR regions; (viii) F(ab′) 2 fragments, a bivalent fragment including two Fab′ fragments linked by a disulfide bridge at the hinge region; (ix) single chain antibody molecules (e.g., single chain Fv; scFv) (Bird et al., 1988; Huston et al., 1988); (x) “diabodies” with two antigen binding sites, comprising a heavy chain variable region (VH) connected to a light chain variable region (VL) in the same polypeptide chain (see, e.g., EP 404,097; WO 93/11161; Hollinger et al., 1993); (xi) “linear antibodies” comprising a pair of tandem Fd segments (VH-CH1-VH-CH1) which, together with complementary light chain polypeptides, form a pair of antigen binding regions (Zapata et al., 1995; U.S. Pat. No. 5,641,870.

“Single-chain variable fragment”, “single-chain antibody variable fragments” or “scFv” antibodies as used herein refers to forms of antibodies comprising the variable regions of only the heavy (VH) and light (VL) chains, connected by a linker peptide. The scFvs are capable of being expressed as a single chain polypeptide. The scFvs retain the specificity of the intact antibody from which it is derived. The light and heavy chains may be in any order, for example, VH-linker-VL or VL-linker-VH, so long as the specificity of the scFv to the target antigen is retained.

An “isolated antibody”, as used herein, can refer to an antibody that is substantially free of other antibodies having different antigenic specificities (e.g., an isolated antibody that specifically binds a TRAIL protein can be substantially free of antibodies that specifically bind antigens other than TRAIL proteins). An isolated antibody that specifically binds a human TRAIL protein can, however, have cross-reactivity to other antigens, such as TRAIL proteins from other species. Moreover, an isolated antibody can be substantially free of other cellular material and/or chemicals.

The terms “monoclonal antibody” or “monoclonal antibody composition” as used herein can refer to a preparation of antibody molecules of single molecular composition. A monoclonal antibody composition displays a single binding specificity and affinity for a particular epitope.

The term “recombinant human antibody”, as used herein, can refer to all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

The term “isotype” can refer to the antibody class (e.g., IgM or IgG1) that is encoded by the heavy chain constant region genes. An antibody can be an immunoglobulin G (IgG), an IgM, an IgE, an IgA or an IgD molecule, or is derived therefrom.

The term “VHH²”, “VHH³” and “VH¹” are representing the heavy chains of three camelid IgG isotypes IgG2, IgG3 and IgG1 respectively. VL¹ is representing the light chain of camelid IgG1. Camelid VL¹ includes, but not limited to Vκ and Vλ.

The term “correspondingly positioned amino acids” and “corresponding amino acids” used herein interchangeably, are amino acid residues that are at an identical position (i.e., they lie across from each other) When two or more amino acid sequences are aligned. Methods for aligning and numbering antibody sequences are well known in the art.

The term “natural” antibody refers to an antibody in which the heavy and light chains of the antibody have been made and paired by the immune system of a multicellular organism. Spleen, lymph nodes, bone marrow, blood and other lymphatic tissues are examples of tissues that contain cells that produce natural antibodies. For example, the antibodies produced by B cells isolated from a first animal immunized with an antigen are natural antibodies. Natural antibodies contain naturally—paired heavy and light chains.

The term “naturally paired” refers to heavy and light chain sequences that have been paired by the immune system of a multi-cellular organism.

The term “mixture”, as used herein, refers to a combination of elements, e.g., cells, that are interspersed and not in any particular order. A mixture is homogeneous and not spatially separated into its different constituents. Examples of mixtures of elements include a number of different cells that are present in the same aqueous solution in a spatially undressed manner.

The term “assessing” includes any form of measurement, and includes determining if an element is present or not. The terms “determining”, “measuring”, “evaluating”, “assessing” and “assaying” are used interchangeably and may include quantitative and/or qualitative determinations. Assessing may be relative or absolute. “Assessing the presence of” includes determining the amount of something present, and/or determining whether it is present or absent.

The term “enriched” is intended to refer to component of a composition (e.g., a particular type of cells) that is more concentrated (e.g., at least 2×, at least 5×, at least 10×, at least 50×, at least 100×, at least 500×, at least 1,000×), relative to other components in the sample (e.g., other cells) than prior to enrichment. In some cases, something that is enriched may represent a significant percent (e.g., greater than 2%, greater than 5%, greater than 10%, greater than 20%, greater than 50%, or more, usually up to about 90%-100%) of the sample in which it resides.

The term “enriching” is intended to any way by which antigen-specific cells can be obtained from a larger population of B cells. As described in greater detail below, enriching may be done by panning, using a bead or cell sorting, for example.

The term “obtaining” in the context of obtaining an element, e.g., cells or sequences, is intended to include receiving the element as well as physically producing the element.

The term “peripheral blood mononucleated cells” or “PBMCs” refers to blood cells that have a single approximately round nucleus (as opposed to a lobed nucleus) and includes lymphocytes (T cells, B cells and NK cells), monocytes and macrophage. PBMCs can be enriched from whole blood using a Ficoll gradient.

The term “antigen-specific B cells” refers to memory B cells that have an antibody that specifically binds to an antigen on their surface, as well as progenitors thereof.

A cell is “derived from” a host if the cell, or the progeny thereof, was obtained from the host. The progeny of a progenitor cell is derived from the progenitor cell.

The term “panning” is used to refer to a method by which B cells are applied to a container (e.g., a plate) that has one or more surfaces that are coated in an antigen or portion thereof. Unbound cells can be removed by washing the surface after the cells are applied to it.

The term “bead-based enrichment” is used to refer to a method by which B cells are mixed with beads, e.g., magnetic beads, that are linked to an antigen or portion thereof.

The term “cell sorting” is used to refer to a method by which B cells are mixed a detectable antigen (e.g., a fluorescently detectable antigen) in solution. In cell sorting methods, cells that are bound to the antigen are sorted from the unbound cells. Fluorescence—activated cell sorting (FACS) is an example of a cell sorting method.

The term “activating” refers to the stimulation of B cells to a) proliferate and b) differentiate into plasma blasts and/or plasma cells and c) secrete antibodies. B cell activation can be done by contacting the B cells with antigen, T cells expressing CD40L and cytokines, although other methods are known (see, e.g., Wykes, Imm. Cell. Biol. 2003 81:328-331).

The term “activated B cells” refers to a cell population that comprises the progeny of a B cell that was activated. As noted above, activation causes B cells to proliferate, and the progeny of such cells are referred to herein as activated B cells.

The term “collecting” refers to the act of separating the cells that in the culture medium from a substrate. Collecting may be done by pipetting or by decanting, for example.

The term “immunized by an antigen” and grammatical equivalents thereof (e.g., “immunizing an animal”) is intended to refer to any animal (humans, rabbits, mice, rats, sheep, cows, chickens, camels) that is mounting an immune response an antigen. An animal may be exposed to a foreign antigen via exposure to an infectious agent, a vaccination, or by administrating an antigen and adjuvant (e.g., by injection), for example. The term “immunized by an antigen” is also intended to include animals that are mounting an immune response against a “self” antigen, i.e., have an autoimmune disease.

The term “lineage rank” refers to the order of lineages when they are listed by their priority factors. The priority factors include but not limited to abundancy of lineage sequences, amplification factor, dynamic change of lineage sequence before and after depleting certain unwanted B cells, dynamic change of lineage sequence abundancy during immunization course, lineages which share the same naïve B-cell origin between VHH and VH, avoidance of developability liability sequences and a combination thereof.

The term “hamming distance” refers to the number of positions at which the corresponding symbols are different between two sequences of equal length.

As used herein, the term “grouped antibodies by lineage”, “lineage-related antibodies” and “antibodies that related by lineage” as well as grammatically equivalent variants thereof, are antibodies that are produced by cells that share a common B cell ancestor. Antibodies that are related by lineage bind to the same epitope of an antigen and are typically very similar in sequence, particularly in their light chain and heavy chain CDR3s. Both the heavy chain and light chain CDR3s of lineage-related antibodies can have an identical length and a near identical sequence (i.e., differ by up to 5, i.e., 0, 1, 2, 3, 4 or 5 residues). Among the group of CDR3s from a lineage, minimal CDR3 distance of a specific CDR3 is the smallest hamming distance of this CDR3 comparing with all other CDR3 of the same length. In some embodiments, the minimal CDR3 distance is equal to or less than 1. In certain cases, the B cell ancestor contains a genome having a rearranged light chain VIC region and a rearranged heavy chain VDJ region, and produces an antibody that has not yet undergone affinity maturation. “Naïve” or “virgin” B cells present in spleen tissue, are exemplary B cell common ancestors.

Related antibodies are related via a common antibody ancestor, e.g., the antibody produced in the naïve B cell ancestor. The term “lineage related antibodies” is intended to describe a group of antibodies that are produced by cells that arise from the same ancestor B-cell. A “lineage group” contains a group of antibodies that are related to one another by lineage.

As used herein, the term “at least the CDR3s” or “at least the CDR3 sequences” refers to only CDR3 sequences, CDR3 sequences in conjunction with CDR1 and/or CDR2 sequences or a sequence of at least 50 contiguous amino acids of the variable domain, up to the entire length of the variable domain, where the sequence contains a CDR3 sequence.

As used herein, the terms “lineage tree” refers to a diagram, resulting from a cladistics analysis, which depicts a hypothetical branching sequence of lineages leading to the individual species of interest. The points of branching within a lineage tree are called nodes.

As used herein, the term “lineage” refers to a theoretical line of descent. Sometimes a group of antibodies related by lineage is referred to as a “lineage group”. The term “lineage” is exclusive, in that a sequence can belong to only one lineage.

As used herein, the term “subgrouping” refers to a further grouping of sequences in a lineage based on unique features or signatures. “Subgroup” is not exclusive, which means one sequence can be in different subgroups. For example, one sequence can have two, three, four, five, or six unique features at the same time. Applying sequence signatures can help to select/narrow-down testing lineages (representative sequences) in a better manner, which may have better biological function/bioactivity outcomes.

As used herein, the term “lineage analysis” refers to the analysis of the theoretical line of descent of an antibody, which is usually done by analyzing a lineage tree.

As used herein, the term “sequence read” refers to a sequence of nucleotides determined by a sequencer, which determination is made, for example, by means of base calling software associated with the technique.

As used herein, the term “obtaining the amino acid sequences” refers to obtaining a file containing amino acid sequences. As is well known, a nucleic acid sequence can be translated into an amino acid sequence in silico.

The phrases “a monoclonal antibody recognizing an epitope on the antigen”, “an antibody recognizing an antigen” and “an antibody specific for an antigen” are used interchangeably herein with the term “an antibody which binds specifically to an antigen” or grammatical equivalents thereof.

The term “specific binding” refers to the ability of an antibody to preferentially bind to a particular antigen that is present in a homogeneous mixture of different molecules. In certain embodiments, a specific binding interaction will discriminate between desirable and undesirable molecules in a sample, in some embodiments more than about 10 to 100 fold or more than e.g., about 1000- or 10,000 fold.

The term “does not substantially bind” to a protein or cells, as used herein, can mean that it cannot bind or does not bind with a high affinity to the protein or cells, i.e., binds to the protein or cells with an K_{D} of 2×10⁻⁶ M or more, more preferably 1×10⁻⁵ M or more, more preferably 1×10⁻⁴ M or more, more preferably 1×10⁻³ M or more, even more preferably 1×10⁻² M or more.

The term “high affinity” for an IgG antibody can refer to an antibody having a K_{D} of 1×10⁻⁶ M or less, preferably 1×10⁻⁷ M or less, more preferably 1×10⁻⁸ M or less, even more preferably 1×10⁻⁹ M or less, even more preferably 1×10⁻¹⁰M or less for a target antigen. However, “high affinity” binding can vary for other antibody isotypes.

The term “rarity score” can refer to a measure of similarity to human germline sequences. In some embodiments, the value is calculated based on framework regions of germlines. Before calculation, a profile of each residue usage percentage in each length of 4 framework regions is determined based on all human IGHV germlines. For each VHH sequence, the residue in each position of 4 framework regions is compared to the profile of that framework region of same length and the rarity score for each position of framework region is calculated based on usage percentage of that residue divided by the top usage percentage of same position. The rarity score for a sequence is average of rarity scores of all framework region residues.

A “humanized antibody” has a sequence that differs from the sequence of an antibody derived from a non-human species by one or more amino acid substitutions, deletions, and/or additions, such that the humanized antibody is less likely to induce an immune response, and/or induces a less severe immune response, as compared to the non-human species antibody, when it is administered to a human subject. In one embodiment, certain amino acids in the framework and constant regions of the heavy and/or light chains of the non-human species antibody are mutated to produce the humanized antibody. In another embodiment, the constant region(s) from a human antibody are fused to the variable region(s) of a non-human species. In another embodiment, a humanized antibody is a CDR grafted antibody comprising one or more CDRs derived from an antibody of a particular species or isotype and the framework of human antibodies. In another embodiment, one or more amino acid residues in one or more CDR sequences of a non-human antibody are changed to reduce the likely immunogenicity of the non-human antibody when it is administered to a human subject, wherein the changed amino acid residues either are not critical for immunospecific binding of the antibody to its antigen, or the changes to the amino acid sequence that are made are conservative changes, such that the binding of the humanized antibody to the antigen is not significantly worse than the binding of the non-human antibody to the antigen. Examples of how to make humanized antibodies may be found in U.S. Pat. Nos. 6,054,297, 5,886,152 and 5,877,293.

The term “chimeric antibody” refers to an antibody that contains one or more regions from one antibody and one or more regions from one or more other antibodies. In one embodiment, one or more of the CDRs are derived from a human antibody. In another embodiment, all of the CDRs are derived from a human antibody. In another embodiment, the CDRs from more than one human antibody are mixed and matched in a chimeric antibody. For instance, a chimeric antibody may comprise a CDR1 from the light chain of a first human antibody, a CDR2 and a CDR3 from the light chain of a second human antibody, and the CDRs from the heavy chain from a third antibody. Other combinations are possible.

The term “biparatopic antibody” refers to an antibody binds to two non-overlapping epitopes of an antigen. In some embodiments, the biparatopic antibody comprises heavy chain only VHHs without light chain. In some embodiments, the biparatopic antibody comprises both heavy chain only VHHs and conventional VH¹/VL¹ pairs. In some embodiments, the biparatopic antibody comprises two conventional VH¹/VL¹ pairs. In some embodiments, the biparatopic antibody has a first heavy chain and a first light chain from a monoclonal antibody targeting one epitope, and an additional antibody heavy chain and light chain targeting another epitope. In some embodiments, the additional light chain or heavy chain can be different from the first light or heavy chains.

The term SAbDab refers to the Structural antibody database at opig.stats.ox.ac.uk/webapps/sabdab).

The binding of an antibody of the disclosed invention to an antigen can be assessed using one or more techniques well established in the art. For example, in some embodiments, an antibody is tested by ELISA assays, for example using a recombinant antigen protein. Still other suitable binding assays include but are not limited to a flow cytometry assay in which the antibody is reacted with a cell line that expresses the human antigen, such as HEK293 cells. Additionally or alternatively, the binding of the antibody, including the binding kinetics (e.g., K_{D} value) can be tested in BIAcore binding assays, Octet Red96 (Pall) and the like.

The term “single B-cell sorting” refers to the sorting of isolated and separated single B cells based on antigen specificity. Technologies for single-cell separation, isolation, and sorting include but are not limited to: FACS (fluorescent activated cell sorting, e.g. using a fluorescent-tagged antigen to isolate cells that bind the antigen), ISAAC (immunospot array assays on a chip), LCM (laser-capture microdissection), microengraving, and droplet microfluidics.

The term “mismatch score” refers to the measure for determining SHM rate. It is calculated as average number of mismatches in 100 bp alignment with the best matched germline gene.

The presently claimed methods are directed to the identification of antibodies that bind to an antigen from affinity maturation in the same B cell lineage as B cells earlier in the lineage that produce antibodies that bind to the antigen.

Thus, in some embodiments, provided is a method of generating an antibody specific for a target antigen. The method comprises:
(a) subject an animal to one or more rounds of immunization with the target antigen;
(b) collecting a first sample of B cells from the animal;
(c) identifying one or more first antibodies from a B cell in the first sample that specifically binds to the antigen and determining a genetic element characterizing the B cell lineage of the B cell in the first sample;
(d) subjecting the animal to one or more additional rounds of immunization with the target antigen;
(e) collecting a second sample of B cells from the animal;
(f) identifying one or more second antibodies from a B cell in the second sample that is from the same B cell lineage as the B cell in the first sample; and
(g) testing the second antibodies for specific binding to the target antigen.

In certain embodiments, the method further comprises repeating steps (d) through (g).

In these methods, antibodies can be generated for any antigen, e.g., a peptide, a protein, a hapten (e.g., conjugated to a carrier molecule), an mRNA, a DNA, a viral vector allowing the expression of an antigen of interest, or a cell.

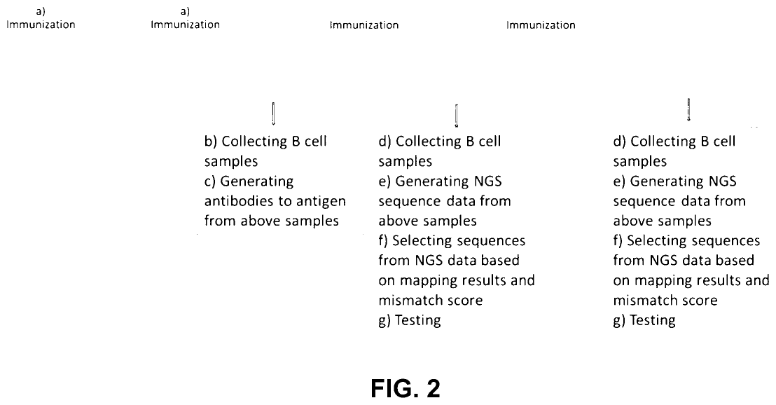

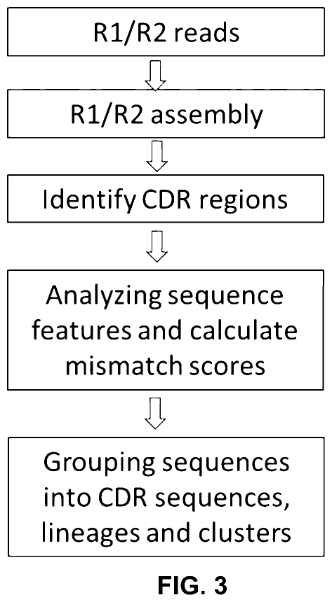

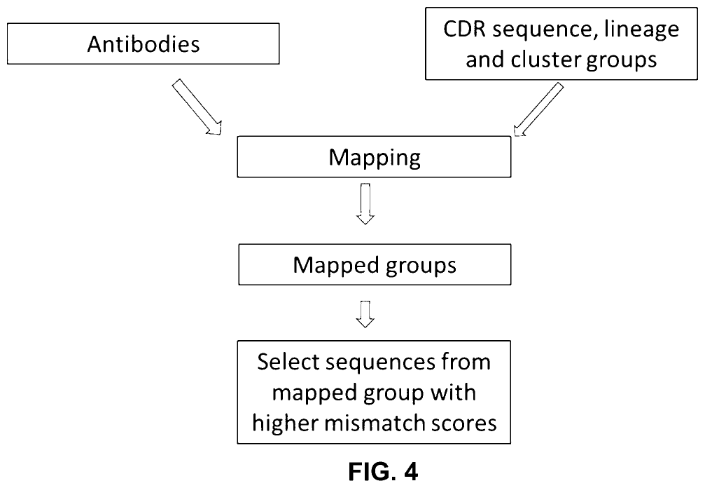

FIG. 2 shows an exemplary timeline and steps for using the claimed method. An animal is immunized multiple times with a target antigen. After initial immunizations, serum PBMCs are collected and the titer of the serum against the target antigen is measured. If the titer is sufficient, then the PBMCs are used to generate antibodies specific to the target antigen using methods like phage display, B cell panning, hybridoma development, NGS, etc. The animal is then further immunized and PBMCs are collected again. NGS data are generated from these PBMC samples and the data is analyzed to calculate mismatch scores and identify groups, as shown in FIG. 3. Sequences in the same groups as and having higher mismatch scores than the first antibodies previously generated are selected for testing (FIG. 4).

The B cells in these methods can be collected by any means known in the art, for example from blood, from a spleen, from lymph nodes, or from bone marrow.

The identifying and sequencing in (c) can be by any means now known or later discovered. In some embodiments, the sequencing is by NGS. Any NGS method can be utilized in these embodiments. See, e.g., Slatko et al. (2018) for an overview of NGS methods.

In various embodiments, B cell samples are enriched against target antigen by any means now known or later discovered, for example using phage display, B cell panning, FACS sorting or other technologies before building NGS libraries and generating NGS data.

Identifying the B cells in a particular lineage (e.g., in (f)) can be by any means now known or later discovered. In some embodiments, the identifying in (f) is by sequencing a portion of a variable region from the B cell and recognizing genetic elements characterizing the B cell lineage. In other embodiments, e.g., where the lineage is too rare to be identified by NGS, the identifying step is by polymerase chain reaction (PCR) of a genetic element characterizing the B cell lineage. In some of these embodiments, the genetic element is a portion of a CDR3 sequence of a heavy chain and/or a light chain of the first antibody. See, e.g., Yaari et al. (2015), Briney et al. (2016) and Kepler et al., (2014) for exemplary methods for identifying and isolating B cell lineages.

NGS technology has been extensively used as an effective sampling method to capture the immune repertoire. With NGS, millions of B cells can be cost effectively sequenced to obtain a good snapshot of the B cell repertoire at a specific time point, and tens of thousands of different antibody sequences can be generated from one typical NGS library. It is an effective sampling method of the immune repertoire and has been used to study the dynamics of immune repertoires and lineage changes therein. In addition, using NGS with primers which amplify antibody sequences with a specific sequence, for example in CDR3, lineage groups of interest from an immune repertoire can be enriched and their changes traced. Using DNA sequences of an antibody derived from NGS, the SHM rate the sequence has accumulated can be estimated by comparing the sequences to germline sequences and discover antibodies with higher activity than the original ones by choosing and testing those sequences with a high SHM rate from the same lineage groups.

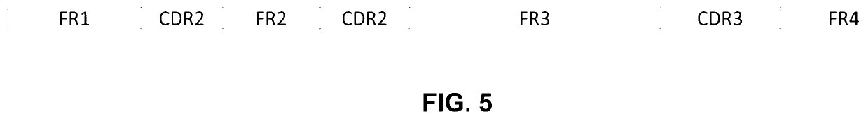

In some embodiments, NGS libraries are built using PCR primers covering partial CDR3 sequences of antibodies identified in step c) to enrich lineage groups for these binders (FIG. 5). Because the immune system is very dynamic, some lineage groups may become very small after some time. To capture those lineages, specially designed PCR primers may be needed to enrich these lineages.

To map a sequence to a group from NGS data, the following criteria may be used: a sequence is mapped to a CDR sequence group if the sequence has same CDR1/CDR2/CDR3 sequences as one of the sequences in the CDR sequence group; a sequence is mapped to a lineage group if the sequence is mapped to same V/J germline genes and has the same CDR3 sequence as one of sequences in the lineage; a sequence is mapped to a cluster group if the sequence has the same CDR3 sequence as one of sequences in the cluster.

Similarly, groups from one NGS dataset can be mapped to groups from another NGS dataset. A CDR sequence group from one NGS dataset can be mapped to a CDR sequence group from another NGS dataset if they share same CDR1/CDR2/CDR3 sequences. A lineage group from one NGS dataset is mapped to a lineage group from another NGS dataset if they are mapped to same V/J germline genes and share one common CDR3. A cluster group from one NGS dataset is mapped to a cluster group from another NGS dataset if they share one common CDR3.

The present methods are not narrowly limited to any particular immunization schedule or time between the collecting in (b) and the collecting in (e). In some embodiments, the collecting in (b) is at least 1 month, 2 months, 3 months, 4 months, 5 months, 6 months, 9 months, 12 months, 18 months, or 24 months before the collecting in (e).

Any type of antibody may be collected using these methods. In some embodiments, the antibody is an immunoglobulin or fragment having two light chains and two heavy chains, or one light chain and one heavy chain. In other embodiments, the antibody is a nanobody.

In some embodiments, additional rounds of immunization, collecting B cells and identifying B cells from a lineage that made antibodies that bound to the target antigen in a previous collection are performed. For purpose of clarification, the term “second antibody” should be broadly interpreted to refer to antibodies identified following these additional rounds of immunization.

Any number of first antibodies and B cell lineages can be identified in these methods. In some embodiments, more than 1, 2, 5, 10, 25, 50, 100, 150 or 200 first antibodies are identified and sequenced in (c).

In some embodiments, binding affinity, specificity and/or neutralizing ability of a first antibody or a second antibody to the antigen is determined.

In various embodiments, a library of B cell sequences is created and sequenced from antibody sequences in B cells before immunization, e.g., to assist in the determination of lineages and to help identify CDR regions.

In additional embodiments, a mismatch score is generated for each first antibody and/or second antibody by aligning sequences encoding each first antibody and/or second antibody to a germline sequence of the animal.

Any of the antibodies identified by the claimed method can be further evaluated to identify favorable features.

In various embodiments, the first antibodies are grouped by CDR sequences, lineages and clusters.

In some embodiments, an enrichment score for each sequence is generated by comparing the frequency of that sequence between two samples. Sequences can be grouped into CDR sequences, e.g., if their CDR1, CDR2 and CDR3 sequences are identical. Additionally, sequences can be further grouped into lineages, e.g., if sequences map to same V/J germline genes and have the same length of CDR3 with maximum one aa difference with CDR3 length longer than 4 and zero difference for CDR3 length equal or shorter than 4, and clusters if sequences have same length of CDR3 with 80% or more identity in CDR3 sequences. Similar enrichment scores for groups are also calculated. Clones that do not show any enrichment in sequences and groups can be filtered out for testing.

In additional embodiments where lineage or lineage rank is considered, lineage priority factors are one or more of lineages from high to low sequences abundancy, lineages from high to low amplification factor, lineages sequences abundancy change during immunization course, lineages sequences abundancy change before and after depleting certain unwanted B cells, lineages which share the same naïve B-cell origin between VHH and VH¹, or avoidance of developability liability sequences.

In further embodiments, a first or second antibody are evaluated for a development liability and eliminated for further development if the liability is present. Nonlimiting examples of development liabilities include unpaired cysteine, N-linked glycosylation, methionine oxidation, tryptophan oxidation, asparagine deamidation, aspartic acid isomerization, lysine glycation, N-terminal glutamates, integrin binding, CD11c, fragmentation, immunogenicity, expression, homogeneity, solubility, stability, viscosity, and/or formulability.

In other embodiments, the antibody is a nanobody. In some of those embodiments, features that indicate an effective nanobody are evaluated by methods described in, e.g., WO 2020/176815 and Applicant's co-pending PCT Patent Application entitled “Selection of Nanobodies Using Sequence Features” filed on Nov. 2, 2023, which are incorporated herein in their entireties. Those features include:
(a) FR2 hydrophilic region;
(b) extended CDR1;
(c) extra disulfide bond between CDR1-CDR3 or FR2-CDR3;
(d) extra disulfide bond within CDR3;
(e) long CDR3 (≥15 aa);
(f) extra disulfide bond within CDR1;
(g) non-classic VHH which have the same V and J germlines as conventional IgG1;
(h) non-classic VHH which have predetermined sequence signatures;
(i) novel canonical binding loop structure;
(j) convergent motif or sequence signature;
(k) a phenylalanine (F) at position 42 (IMGT numbering);
(l) a short hinge;
(m) two or more cysteines in the nanobody sequence;
(n) a glutamine (Q) at position 123 (IMGT numbering);
(o) low immunogenicity metric;
(p) non-classic VHH derived from germline IGHV3;
(q) non-classic VHH derived from germline IGHV4;
(r) a histidine (H), aspartic acid (D) or glutamic acid (E) in the CDR region;
(s) a histidine (H), aspartic acid (D) or glutamic acid (E) in the first three amino acid residues, the FR2 region, or the first sixteen amino acid residues of the FR3 region of the nanobody sequence;
(t) a tyrosine (Y) at position 42 (IMGT numbering), and the nanobody having a loop, concave paratope structure configuration; or
(u) a phenylalanine (F) at position 42 (IMGT numbering), and the nanobody having a convex paratope structure configuration.

In these methods, any of the antibodies can be expressed by any means known in the art. In some embodiments, the selected antibody is expressed in prokaryotic cells. In other embodiments, the selected antibody is expressed in eukaryotic cells.

Preferred embodiments are described in the following examples. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims, which follow the examples.

### Example 1: High Affinity Nanobodies Against PD1 Discovered Through Longitudinal Lineage Tracing

Using PD1 recombinant protein as immunogen, one alpaca was immunized multiple times. After the seventh immunization, PBMC from the animal were collected and several nanobodies (in VHH format) were identified from the PBMC using phage display technology. Three of these binders (Table 1) showed a variety of binding activities belonging to different lineage groups.

TABLE 1
Three binders against PD1

| Name | Mismatch score | CDR3 | Ka | Kd | KD |
|---|---|---|---|---|---|
| 1173 | 10 | AADGDASDISYAPPRDYEYDY | 3.73E+04 | 2.21E−04 | 5.92E−09 |
| | | (SEQ ID NO: 1) | | | |
| 1182 | 10 | NAGAPPPGGLGYDESDY | 1.19E+04 | 1.68E−05 | 1.42E−09 |
| | | (SEQ ID NO: 2) | | | |
| 1194 | 10 | GVDRRQYGLGIPPLADH | 1.31E+05 | 8.83E−05 | 6.77E−10 |
| | | (SEQ ID NO: 3) | | | |

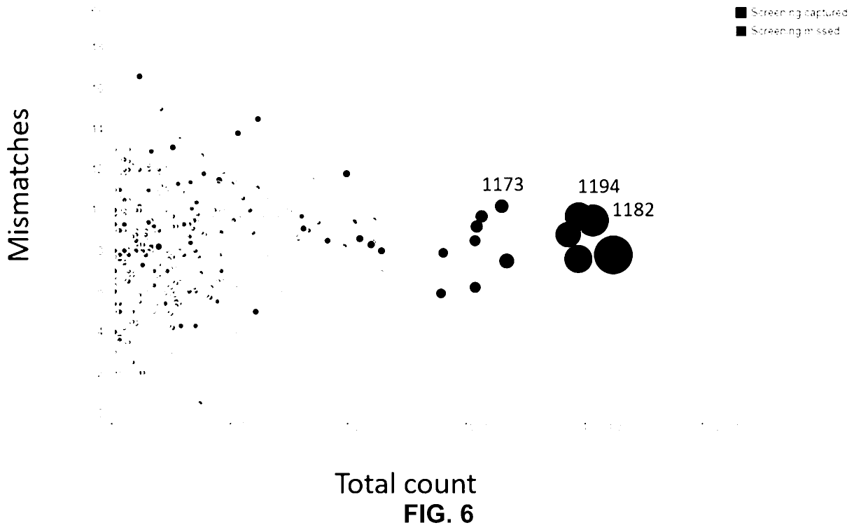

Several NGS datasets were generated from PBMC samples collected after the 7th, 20th and 24th immunizations. FIG. 6 shows lineage groups and clone mapped groups from the NGS data generated after the 7th immunization. In this specific case, 85 clones identified using phage display technology were mapped to 26 lineage groups (darker dots).

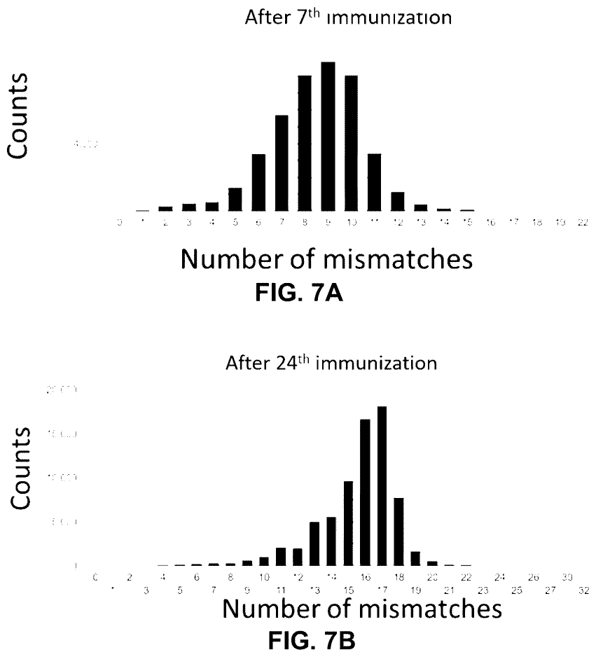

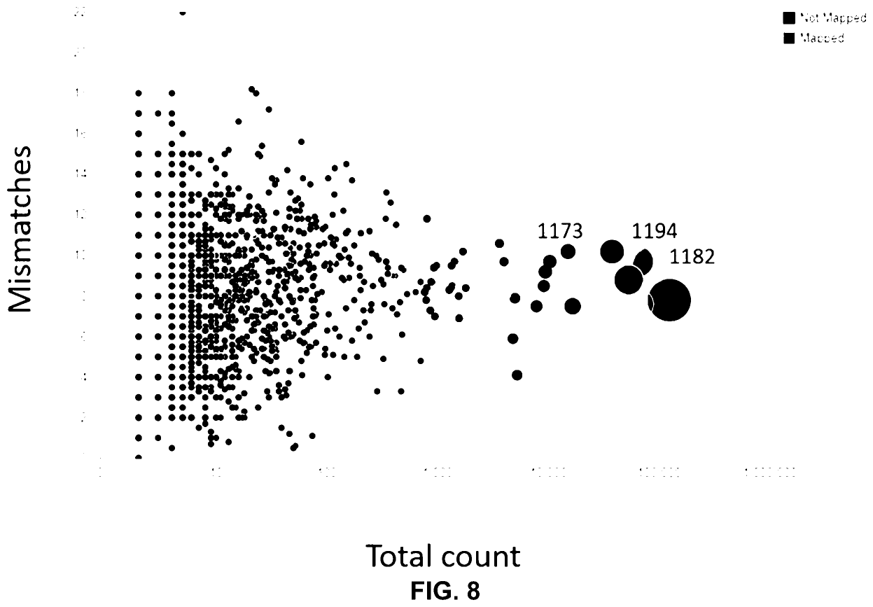

Three lineage groups mapped by three clones in Table 1 were labeled by a corresponding clone name. With further immunization, we determined whether, as expected, antibody sequences accumulate more and more mutations. Comparing NGS dataset generated after 7th immunization to those generated after 24th immunization, significant increases of mismatch scores were observed (FIG. 7). To perform longitudinal lineage tracing, we also performed mapping between two NGS datasets. FIG. 8 shows an example of such mapping results. As expected, the immune repertoire is very dynamic, and many lineages disappeared while many new ones showed up. Table 2 shows the frequency change for some shared lineages after further immunization.

TABLE 2
Frequency changes of some shared lineage groups from FIG. 8

| Shared | Frequency in | Frequency in |
|---|---|---|
| lineage | ALP07VHH_PB_PAN | NBL504-A27L1P1L-R1 |
| 1 | 14.1% | 19.3% |
| 2 | 10.8% | 5.2% |
| 3 | 10.7% | 0.01% |
| 4 | 1.5% | 8.1% |
| 5 | 0.69% | 0.09% |
| 6 | 0.26% | 14.1% |
| 7 | 0.17% | 0.003% |
| 8 | 0.09% | 0.04% |

To discover possible better antibodies than the three antibodies in Table 1, we mapped these three clones to groups in various NGS datasets, and clones with high mismatch scores were selected and tested. The results are shown in Table 3. The SPR results are shown in Table 4. In all three cases, about a 5-fold affinity increase was observed, based on the KD value. Improved blocking activity was also observed in two cases, based on IC50 values.

TABLE 3
Clone selection for three binders

| | Mismatch | | | Mismatch | |
|---|---|---|---|---|---|
| Name | score | CDR3 | New clones | score | CDR3 |
| 1173 | 10 | AADGDASDISY | NBL504-A1B2- | 13 | AADGDASDISYAPPR |
| | | APPRDYEYDY | S3_98304 | | DYEYDY |
| | | (SEQ ID NO: 1) | (NBL504#100) | | (SEQ ID NO: 4) |
| 1182 | 10 | NAGAPPPGGL | NBL504-A1B3- | 16 | NAGAPPSGGYGFDES |
| | | GYDESDY | S11_3367 | | DY |
| | | (SEQ ID NO: 2) | (NBL504#103) | | (SEQ ID NO: 5) |
| 1194 | 10 | GVDRRQYGLG | NBL504-A1B3- | 19 | GVDTRQYGLGIPPLA |
| | | IPPLADH | S11_1519 | | DH (SEQ ID NO: 6)/ |
| | | | (NBL504#101)/ | | GIDRRQYGLGIPPLAD |
| | | | NBL504-A1B3- | | H (SEQ ID NO: 7) |
| | | (SEQ ID NO: 3) | S11_35819 | | |
| | | | (NBL504#102) | | |

TABLE 4
SPR and blocking results for selected clones

| | | | | Blocking | | | | | Blocking |
|---|---|---|---|---|---|---|---|---|---|
| Name | Ka | Kd | KD | IC50 (nM) | New clone | Ka | Kd | KD | IC50 (nM) |
| 1173 | 3.73E+04 | 2.21E−04 | 5.92E−09 | | NBL504-A1B2- | 8.36E+04 | 7.94E−05 | 9.50E−10 | |
| | | | | | S3_98304 | | | | |
| | | | | | (NBL504#100) | | | | |
| 1182 | 1.19E+04 | 1.68E−05 | 1.42E−09 | 6.98 | NBL504-A1B3- | 6.85E+04 | 1.91E−05 | 2.78E−10 | 3.68 |
| | | | | | S11_3367 | | | | |
| | | | | | (NBL504#103) | | | | |
| 1194 | 1.31E+05 | 8.83E−05 | 6.77E−10 | 9.69 | NBL504-A1B3- | 2.83E+05 | 7.05E−05 | 2.49E−10 | 2.68 |
| | | | | | S11_1519 | | | | |
| | | | | | (NBL504#101) | | | | |
| | | | | | NBL504-A1B3- | 2.98E+05 | 4.63E−05 | 1.55E−10 | 2.89 |
| | | | | | S11_35819 | | | | |
| | | | | | (NBL504#102) | | | | |

### Example 2: Changes of Mismatch Scores Through Longitudinal Analysis and Correlation with Affinity

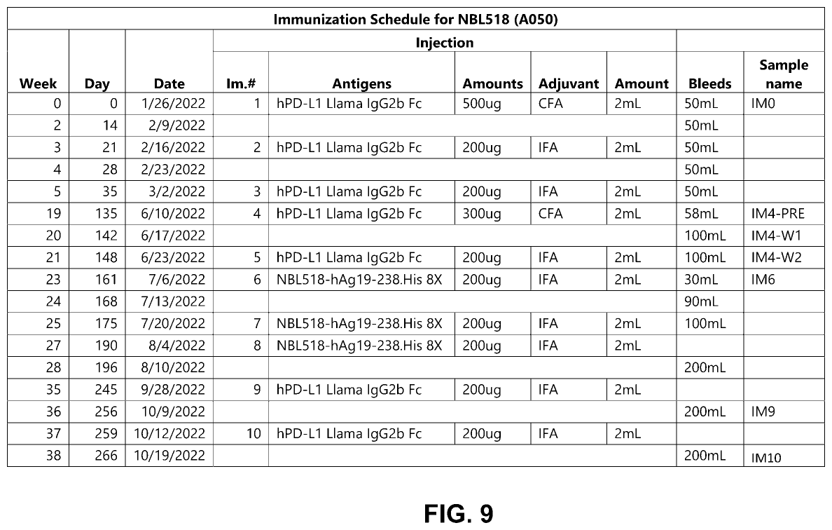

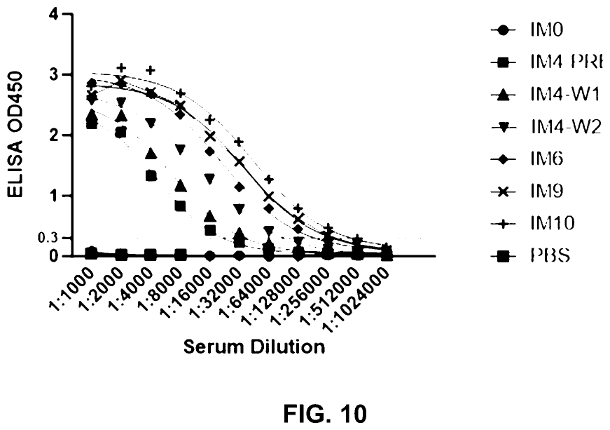

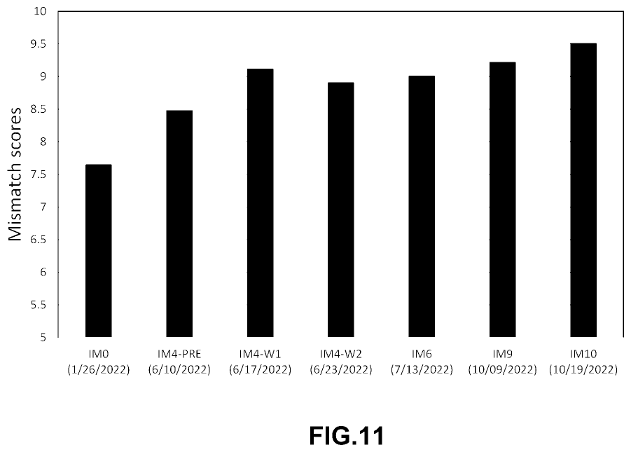

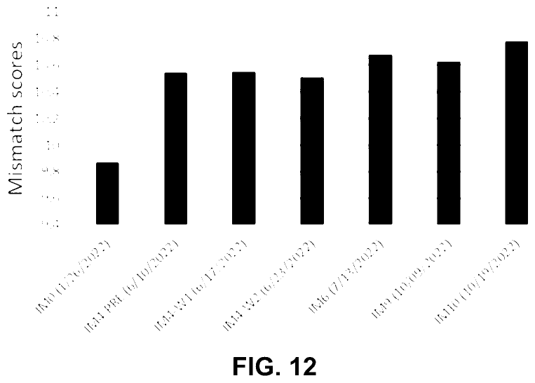

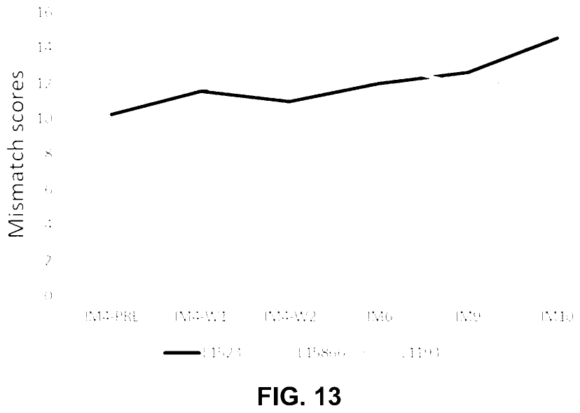

To discover binders for PD-L1 target (NBL518 project), an alpaca (A050) was immunized with human PD-L1 immunogens. FIG. 9 shows detailed immunization and bleeding schedules for this animal. To avoid any repertoire bias caused by experimental panning/enrichment procedures, PBMC samples from some bleeds (FIG. 9) were directly used to construct NGS libraries. The titers of these PBMC samples showed gradual increase with more immunization (FIG. 10). Average mismatch scores for each library were calculated based on NGS sequences. FIG. 11 showed that all samples after immunization had higher average mismatch scores as compared to samples before immunization and there is a gradual increase trend as more immunizations were performed. To analyze mismatch score changes in lineages, we looked at common lineages in these samples. A total of 206 lineages were found to be shared among these samples and average mismatch scores of these lineages showed gradual increase (FIG. 12). To further focus on antigen specific lineages, e.g., lineages contain antibodies binding to the target, we identified three such lineages shared among samples after immunization. FIG. 13 shows the dynamics of mismatch score changes of these three lineages. Overall there is gradual increase of mismatch scores with more immunization, similar to the results shown in FIG. 11 and FIG. 12.

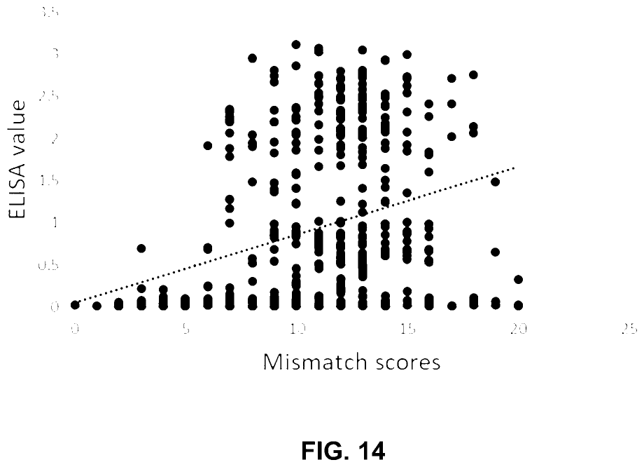

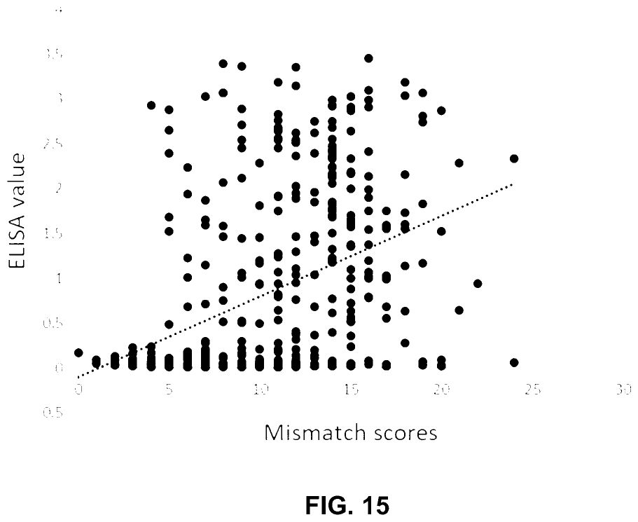

Many binders with various affinities for PD-L1 targets were discovered from this animal through several discovery campaigns. A low but statistically significant correlation (P<0.05) was observed between mismatch scores and ELISA value of these binders (FIG. 14). Similar result was observed from binders identified from a llama (FIG. 15). Such results again suggest that affinities increase with more somatic mutations.

### EMBODIMENTS

Embodiment 1. A method of generating an antibody specific for a target antigen, the method comprising
(a) subject an animal to one or more rounds of immunization with the target antigen;
(b) collecting a first sample of B cells from the animal;
(c) identifying one or more first antibodies from a B cell in the first sample that specifically bind to the antigen and determining a genetic element characterizing the B cell lineage of the B cell in the first sample;
(d) subjecting the animal to one or more additional rounds of immunization with the target antigen;
(e) collecting a second sample of B cells from the animal;
(f) identifying one or more second antibodies from a B cell in the second sample that is from the same B cell lineage as the B cell in the first sample; and
(g) testing the second antibodies for specific binding to the target antigen.

Embodiment 2. The method of embodiment 1, further comprising
(h) repeating steps (d) through (g).

Embodiment 3. The method of embodiment 1 or 2, wherein the identifying in
(c) comprises sequencing the first antibodies by NGS.

Embodiment 4. The method of any one of embodiments 1-3, wherein the identifying in (c) comprises B cell panning and/or phage display.

Embodiment 5. The method of any one of embodiments 1-4, wherein the B cell in the second sample in (f) recognizes the genetic element characterizing the B cell lineage.

Embodiment 6. The method of embodiment 5, wherein the genetic element is a portion of a variable region of the first antibody.

Embodiment 7. The method of embodiment 6, wherein the genetic element is a portion of a CDR3 sequence of a heavy chain and/or a light chain of the first antibody.

Embodiment 8. The method of any one of embodiments 1-4, wherein the identifying in (f) comprises sequencing the second antibodies by NGS.

Embodiment 9. The method of embodiment 1, wherein the identifying in (f) comprises polymerase chain reaction (PCR) of the genetic element characterizing the B cell lineage.

Embodiment 10. The method of embodiment 9, wherein the genetic element is a portion of a variable region of the first antibody.

Embodiment 11. The method of embodiment 10, wherein the genetic element is a portion of a CDR3 sequence of a heavy chain and/or a light chain of the first antibody.

Embodiment 12. The method of any one of embodiments 1-11, wherein the collecting in (b) is at least 1 month, 2 months, 3 months, 4 months, 5 months, 6 months, 9 months, 12 months, 18 months, or 24 months before the collecting in (e).

Embodiment 13. The method of any one of embodiments 1-12, wherein the first antibody or the second antibody is an immunoglobulin or a nanobody.

Embodiment 14. The method of any one of embodiments 1-13, wherein the animal is a mammal.

Embodiment 15. The method of embodiment 14, wherein the mammal is a camelid.

Embodiment 16. The method of any one of embodiments 1-15, wherein two or more first antibodies are identified in (c).

Embodiment 17. The method of embodiment 16, wherein the first antibodies are grouped by CDR sequences, lineages and clusters.

Embodiment 18. The method of embodiment 16 or 17, wherein a mismatch score is generated for each first antibody and/or second antibody by aligning sequences encoding each first antibody and/or second antibody to a germline sequence of the animal.

Embodiment 19. The method of embodiment 18, wherein the second antibody having a higher mismatch score is prioritized for testing in (g).

Embodiment 20. The method of any one of embodiments 17-19, wherein sequences in a CDR group have the same CDR1, CDR2 and CDR3 sequences.

Embodiment 21. The method of any one of embodiments 17-19, wherein sequences in a lineage map to the same V and J germline genes and have a maximum distance of a specific CDR3 equal to or less than 1 aa between the closest two CDR3s in the lineage, wherein all CDR3s have the same length.

Embodiment 22. The method of any one of embodiments 17-19, wherein sequences in a cluster have the same CDR3 length with CDR3 identity larger than 80% between the closest two CDR3s in the cluster.

Embodiment 23. The method of any one of embodiments 17-22, wherein the first antibody or the second antibody is a nanobody.

Embodiment 24. The method of any one of embodiments 1-23, wherein antibodies are prioritized by at least one of the following features:
(a) FR2 hydrophilic region;
(b) extended CDR1;
(c) extra disulfide bond between CDR1-CDR3 or FR2-CDR3;
(d) extra disulfide bond within CDR3;
(e) long CDR3 (≥15 aa);
(f) extra disulfide bond within CDR1;
(g) non-classic VHH which have the same V and J germlines as conventional IgG1;
(h) non-classic VHH which have predetermined sequence signatures;
(i) novel canonical binding loop structure;
(j) convergent motif or sequence signature;
(k) a phenylalanine (F) at position 42 (IMGT numbering);
(l) a short hinge;
(m) two or more cysteines in the nanobody sequence;
(n) a glutamine (Q) at position 123 (IMGT numbering);
(o) low immunogenicity metric;
(p) non-classic VHH derived from germline IGHV3;
(q) non-classic VHH derived from germline IGHV4;
(r) a histidine (H), aspartic acid (D) or glutamic acid (E) in the CDR region;
(s) a histidine (H), aspartic acid (D) or glutamic acid (E) in the first three amino acid residues, the FR2 region, or the first sixteen amino acid residues of the FR3 region of the nanobody sequence;
(t) a tyrosine (Y) at position 42 (IMGT numbering), and the nanobody having a loop, concave paratope structure configuration; or
(u) a phenylalanine (F) at position 42 (IMGT numbering), and the nanobody having a convex paratope structure configuration.

Embodiment 25. The method of any one of embodiments 1-24, wherein the first antibody or the second antibody is eliminated if the antibody has at least one development liability, wherein the at least one development liability is immunogenicity, expression, homogeneity, solubility, stability, viscosity or formulability.

Embodiment 26. The method of any one of embodiments 1-24, wherein the first antibody or the second antibody is tested for binding with a wide range of affinities.

Embodiment 27. The method of any one of embodiments 1-26, wherein the animal is immunized with a peptide, a protein, an mRNA, a DNA, a viral vector or a cell.

Embodiment 28. The method of any one of embodiments 1-27, wherein binding affinity, specificity, or neutralizing ability of the first antibody or the second antibody to the antigen is determined.

Embodiment 29. The method of any one of embodiments 1-28, wherein the first antibody or the second antibody is expressed in prokaryotic cells.

Embodiment 30. The method of any one of embodiments 1-28, wherein a first antibody or a second antibody is expressed in eukaryotic cells.

In view of the above, it will be seen that several objectives of the invention are achieved and other advantages attained.

As various changes could be made in the above methods and compositions without departing from the scope of the invention, it is intended that all matter contained in the above description and shown in the accompanying drawings shall be interpreted as illustrative and not in a limiting sense.

All references cited in this specification, including but not limited to patent publications and non-patent literature, and references cited therein, are hereby incorporated by reference. The discussion of the references herein is intended merely to summarize the assertions made by the authors and no admission is made that any reference constitutes prior art. Applicants reserve the right to challenge the accuracy and pertinence of the cited references.

As used herein, in particular embodiments, the terms “about” or “approximately” when preceding a numerical value indicates the value plus or minus a range of 10%. Where a range of values is provided, it is understood that each intervening value, to the tenth of the unit of the lower limit unless the context clearly dictates otherwise, between the upper and lower limit of that range and any other stated or intervening value in that stated range is encompassed within the disclosure. That the upper and lower limits of these smaller ranges can independently be included in the smaller ranges is also encompassed within the disclosure, subject to any specifically excluded limit in the stated range. Where the stated range includes one or both limits, ranges excluding either or both of those included limits are also included in the disclosure.

The indefinite articles “a” and “an,” as used herein in the specification and in the embodiments, unless clearly indicated to the contrary, should be understood to mean “at least one.”

The phrase “and/or,” as used herein in the specification and in the embodiments, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements can optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the embodiments, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the embodiments, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the embodiments, shall have its ordinary meaning as used in the field of patent law.

As used herein in the specification and in the embodiments, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements can optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

### REFERENCES

Arbabi-Ghahroudi. Camelid Single-Domain Antibodies: Historical Perspective and Future Outlook. Front. Immunol., 20 Nov. 2017.
Basilico, et al. Four individually druggable MET hotspots mediate HGF-driven tumor progression, The Journal of Clinical Investigation, Volume 124 Number 7 Jul. 2014.
Bird et al., Science 242:423-426 (1988).
Briney et al., Scientific Reports 6:23901 (2016).
Conrath K E, et al. Emergence and evolution of functional heavy-chain antibodies in Camelidae. Dev Comp Immunol 27:87-103, 2003.
Daley L P, Clin. Vaccine Immunol. 17:239-46, 2010.
Daniela Bumbaca et al., Highly specific off-target binding identified and eliminated during the humanization of an antibody against FGF receptor 4. mAbs 3:4, 376-386; July/August 2011.
DeKosky, B. J. et al., High-throughput sequencing of the paired human immunoglobulin heavy and light chain repertoire. Nature Biotechnology, 31 (2), 166-169 (2013).
Deschacht, et al. A Novel Promiscuous Class of Camelid Single-Domain Antibody Contributes to the Antigen-Binding Repertoire, The Journal of Immunology. 184 (10) 5696-5704 May 2010.
Griffin et al. Analysis of heavy and light chain sequences of conventional camelid antibodies from Camelus dromedarius and Camelus bactrianus species, Journal of Immunological Methods Volume 405, Pages 35-46, March 2014.
Hollinger et al., Proc. Natl. Acad. Sci. USA, 90:6444-6448 (1993).
Huston et al., PNAS (USA) 85:5879-5883 (1988).
Jiang et al., Sci. Trans. Med 5:171ra19 (2013).
Kepler et al., Front. Immunol. 5:170 (2014).
Klarenbeek, et al. Camelid Ig V genes reveal significant human homology not seen in therapeutic target genes, providing for a powerful therapeutic antibody platform, mAbs 7:4, 693-706; 2015.
Küppers R. Mechanisms of B-cell lymphoma pathogenesis. Nat Rev Cancer (2005) 5:251-262.
McCoy L E, et al. Potent and broad neutralization of HIV-1 by a llama antibody elicited by immunization. J. Exp. Med. 2012.
Nguyen et al. Camel heavy-chain antibodies: diverse germline VHH and specific mechanism enlarge the antigen-binding repertoire The EMBO Journal 19 No. 5 2000.
Nguyen et al. Heavy-chain antibodies in Camelidae; a case of evolutionary innovation. Immunogenetics 54:39-47, 2002.
Pan, X., López Acevedo, S. N., Cuziol, C., de Tavernier, E., Fahad, A. S., Longjam, P. S., Rao, S. P., Aguilera-Rodríguez, D., Rezé, M., Bricault, C. A., Gutiérrez-González, M. F., de Souza, M. O., DiNapoli, J. M., Vigne, E., Shahsavarian, M. A., & DeKosky, B. J. (2023). Large-scale antibody immune response mapping of splenic B cells and bone marrow plasma cells in a transgenic mouse model. Frontiers in Immunology, 14.
Phad, G. E. et al., Clonal structure, stability and dynamics of human memory B cells and circulating plasmablasts. Nature Immunology, 23 (7), 1-10 (2022).
Slatko et al, Curr. Protoc. Mol. Biol. 122: e59, 2018.
Ward et al., Nature 341, 544-546 (1989).
Woninga, et al. DNA immunization combined with scFv phage display identifies antagonistic GCGR specific antibodies and reveals new epitopes on the small extracellular loops, MABS, VOL. 8, NO. 6, 1126-1135, 2016.
Yaari, G. and Kleinstein, S. Genome Med. 7:121 (2015).
Zapata et al. Protein Eng. 8 (10): 1057-1062 (1995).
Zhang et al., Immunol. Rev. 270:8-19 (2016).
European Patent No. 404,097.
PCT Patent Publication WO 93/11161.
PCT Patent Publication WO 2020/176815.
PCT Patent Publication WO 2020/208555.
U.S. Patent Application Publication 2019/0065677A1.
U.S. Pat. No. 5,641,870.
U.S. Pat. No. 6,054,297.
U.S. Pat. No. 5,886,152.
U.S. Pat. No. 5,877,293.
U.S. Pat. No. 7,117,096 B2.
U.S. Pat. No. 7,432,063 B2.
U.S. Pat. No. 10,101,333 B2.

What is claimed is:

1. A method of generating an antibody specific for a target antigen, the method comprising
(a) subject an animal to one or more rounds of immunization with the target antigen;
(b) collecting a first sample of B cells from the animal;
(c) identifying one or more first antibodies from a B cell in the first sample that specifically bind to the antigen and determining a genetic element characterizing the B cell lineage of the B cell in the first sample;
(d) subjecting the animal to one or more additional rounds of immunization with the target antigen;
(e) collecting a second sample of B cells from the animal;
(f) identifying one or more second antibodies from a B cell in the second sample that is from the same B cell lineage as the B cell in the first sample; and
(g) testing the second antibodies for specific binding to the target antigen.

2. The method of claim 1, further comprising (h) repeating steps (d) through (g).

3. The method of claim 1 or 2, wherein the identifying in (c) comprises sequencing the first antibodies by NGS.

4. The method of any one of claims 1-3, wherein the identifying in (c) comprises B cell panning and/or phage display.

5. The method of any one of claims 1-4, wherein the B cell in the second sample in (f) recognizes the genetic element characterizing the B cell lineage.

6. The method of claim 5, wherein the genetic element is a portion of a variable region of the first antibody.

7. The method of claim 6, wherein the genetic element is a portion of a CDR3 sequence of a heavy chain and/or a light chain of the first antibody.

8. The method of any one of claims 1-4, wherein the identifying in (f) comprises sequencing the second antibodies by NGS.

9. The method of claim 1, wherein the identifying in (f) comprises polymerase chain reaction (PCR) of the genetic element characterizing the B cell lineage.

10. The method of claim 9, wherein the genetic element is a portion of a variable region of the first antibody.

11. The method of claim 10, wherein the genetic element is a portion of a CDR3 sequence of a heavy chain and/or a light chain of the first antibody.

12. The method of any one of claims 1-11, wherein the collecting in (b) is at least 1 month, 2 months, 3 months, 4 months, 5 months, 6 months, 9 months, 12 months, 18 months, or 24 months before the collecting in (e).

13. The method of any one of claims 1-12, wherein the first antibody or the second antibody is an immunoglobulin or a nanobody.

14. The method of any one of claims 1-13, wherein the animal is a mammal.

15. The method of claim 14, wherein the mammal is a camelid.

16. The method of any one of claims 1-15, wherein two or more first antibodies are identified in (c).

17. The method of claim 16, wherein the first antibodies are grouped by CDR sequences, lineages and clusters.

18. The method of claim 16 or 17, wherein a mismatch score is generated for each first antibody and/or second antibody by aligning sequences encoding each first antibody and/or second antibody to a germline sequence of the animal.

19. The method of claim 18, wherein the second antibody having a higher mismatch score is prioritized for testing in (g).

20. The method of any one of claims 17-19, wherein sequences in a CDR group have the same CDR1, CDR2 and CDR3 sequences.

21. The method of any one of claims 17-19, wherein sequences in a lineage map to the same V and J germline genes and have a maximum distance of a specific CDR3 equal to or less than 1 aa between the closest two CDR3s in the lineage, wherein all CDR3s have the same length.

22. The method of any one of claims 17-19, wherein sequences in a cluster have the same CDR3 length with CDR3 identity larger than 80% between the closest two CDR3s in the cluster.

23. The method of any one of claims 17-22, wherein the first antibody or the second antibody is a nanobody.

24. The method of any one of claims 1-23, wherein antibodies are prioritized by at least one of the following features:
(a) FR2 hydrophilic region;
(b) extended CDR1;
(c) extra disulfide bond between CDR1-CDR3 or FR2-CDR3;
(d) extra disulfide bond within CDR3;
(e) long CDR3 (≥15 aa);
(f) extra disulfide bond within CDR1;
(g) non-classic VHH which have the same V and J germlines as conventional IgG1;
(h) non-classic VHH which have predetermined sequence signatures;
(i) novel canonical binding loop structure;
(j) convergent motif or sequence signature;
(k) a phenylalanine (F) at position 42 (IMGT numbering);
(l) a short hinge;
(m) two or more cysteines in the nanobody sequence;
(n) a glutamine (Q) at position 123 (IMGT numbering);
(o) low immunogenicity metric;
(p) non-classic VHH derived from germline IGHV3;
(q) non-classic VHH derived from germline IGHV4;
(r) a histidine (H), aspartic acid (D) or glutamic acid (E) in the CDR region;
(s) a histidine (H), aspartic acid (D) or glutamic acid (E) in the first three amino acid residues, the FR2 region, or the first sixteen amino acid residues of the FR3 region of the nanobody sequence;
(t) a tyrosine (Y) at position 42 (IMGT numbering), and the nanobody having a loop, concave paratope structure configuration; or
(u) a phenylalanine (F) at position 42 (IMGT numbering), and the nanobody having a convex paratope structure configuration.

25. The method of any one of claims 1-24, wherein the first antibody or the second antibody is eliminated if the antibody has at least one development liability, wherein the at least one development liability is immunogenicity, expression, homogeneity, solubility, stability, viscosity or formulability.

26. The method of any one of claims 1-24, wherein the first antibody or the second antibody is tested for binding with a wide range of affinities.

27. The method of any one of claims 1-26, wherein the animal is immunized with a peptide, a protein, an mRNA, a DNA, a viral vector or a cell.

28. The method of any one of claims 1-27, wherein binding affinity, specificity, or neutralizing ability of the first antibody or the second antibody to the antigen is determined.

29. The method of any one of claims 1-28, wherein the first antibody or the second antibody is expressed in prokaryotic cells.

30. The method of any one of claims 1-28, wherein a first antibody or a second antibody is expressed in eukaryotic cells.


SEQUENCE LISTING (7 sequences)

SEQ ID NO: 1 - protein, 21 aa, synthetic construct
AADGDASDISYAPPRDYEYDY

SEQ ID NO: 2 - protein, 17 aa, synthetic construct
NAGAPPPGGLGYDESDY

SEQ ID NO: 3 - protein, 17 aa, synthetic construct
GVDRRQYGLGIPPLADH

SEQ ID NO: 4 - protein, 21 aa, synthetic construct
AADGDASDISYAPPRDYEYDY

SEQ ID NO: 5 - protein, 17 aa, synthetic construct
NAGAPPSGGYGFDESDY

SEQ ID NO: 6 - protein, 17 aa, synthetic construct
GVDTRQYGLGIPPLADH

SEQ ID NO: 7 - protein, 17 aa, synthetic construct
GIDRRQYGLGIPPLADH